(19) United States
(12) Patent Application Publication
(10) Pub. No.: US 2004/0241237 A1
(43) Pub. Date: Dec. 2, 2004

(54) Porous carbon material

(76) Inventors: Jean-Paul Pirard, Chenee (BE); Rene Pirard, Liege (BE); Nathalie Job, Liege (BE)

Correspondence Address: JACOBSON HOLMAN PLLC, 400 SEVENTH STREET N.W., SUITE 600, WASHINGTON, DC, 20004, US

(21) Appl. No.: 10/484,691

(22) PCT Filed: Jul. 18, 2002
(86) PCT No.: PCT/EP02/08073

(30) Foreign Application Priority Data
Jul. 26, 2001 (EP) 01202862.7

(51) Int. Cl. A61K 9/14

(52) U.S. Cl. 424/484

(57) ABSTRACT

The invention concerns a method for preparing an organic gel metal-doped or not comprising a mixture in a solvent of an hydroxylated benzene with an aldehyde to form a gel whereof the texture is regulated by adjusting the pH of the starting mixture. The invention also concerns a method for preparing a carbonaceous material, metal-doped or not and obtained by drying followed by pyrolysis of the corresponding organic gel, whereof the porous texture is regulated according to the pH of the starting mixture. The resulting carbonaceous materials can be used in chemical catalysis or for making fuel cell electrodes.

The invention relates to an organic gel with a determined porous texture, in particular an organic gel charged with metal, and a method of preparing them. The invention also relates to a porous carbon material, in particular a porous carbon material charged with metal, and a method of preparing them.

Finally, the invention relates to the use of the porous carbon material obtained in catalysis or in the fabrication of fuel cell electrodes.

Porous carbon materials in the form of carbon foam, aerogel or xerogel are known in the art.

The term carbon foam is used to designate a porous material of low density that is characterised by a dispersion of gas in a solid or a liquid. The foam can be a closed cell foam or an open cell foam. Open cell foams include xerogels and aerogels.

Aerogels are very similar to xerogels. They are produced by drying a traditional gel. They are porous carbon materials of high specific surface area comprising micropores (pore size less than 2 nm), mesopores (pore size from 2 to 50 nm), and macropores (pore size greater than 50 nm). However, in U.S. Pat. No. 5,945,084, M. W. Droege describes xerogels obtained by drying under non-supercritical conditions as more dense than aerogels obtained by supercritical drying. Xerogels are characterised by a reduction of the number of macropores and mesopores, often attributed to a shrunken texture that appears on drying by evaporating the solvent.

Porous carbon materials are generally obtained from an organic hydroxylated benzene-aldehyde gel. Hydroxylated benzene-aldehyde gels are obtained by polycondensation of a hydroxylated benzene with an aldehyde. They are then dried conventionally in a supercritical manner or by transfer of solvent, followed by pyrolysis in an inert atmosphere to form a porous texture carbon material.

To impart sufficient mechanical strength to a porous carbon material, whilst at the same time retaining sufficient permeability to gases, it is important to control the specific surface area, porous texture, density and distribution of pore sizes, that is to say to fix the proportions in which micropores, mesopores and macropores must be distributed. In other words, it is important to control the porous texture of the material. The definition and characterisation of the porous texture used here are those commonly recognised and described by A. J. Lecloux in his paper Texture of Catalysts, published in Catalysis Science and Technology by R. Anderson and M. Boudart in 1981.

We have now found that by adjusting the pH of the mixture in an appropriate range at the outset, it is possible to control not only the texture of the initial organic gel obtained from the starting mixture and the intermediate gel obtained after drying, but also the final carbon material obtained after pyrolysis, whether charged with metal or not.

In a first aspect, the present invention consists in a method of preparing an organic gel with a determined porous texture comprising mixing hydroxylated benzene with an aldehyde in a solvent to form a gel, which method is characterised in that the texture of the gel is regulated by adjusting the pH of the starting mixture.

The hydroxylated benzene of the invention is, for example, phenol, resorcinol, catechol, hydroquinone, phloroglucinol, polyhydroxybenzene or mixtures thereof. Resorcinol or 1,3-dihydroxybenzene is preferably chosen.

The aldehyde of the invention is, for example, formaldehyde, glyoxal, glutaraldehyde, furfural or mixtures thereof. The starting aldehydes may also be in aqueous solution or in solution in some other solvent.

With resorcinol as the hydroxylated benzene, formaldehyde is preferably chosen to form a mixture of products of condensation and addition.

The hydroxylated benzene and the aldehyde can be mixed in varying proportions at any temperature from the freezing point to the boiling point of the solvent.

In the case of mixing resorcinol and formaldehyde, which can be effected at ambient temperature, a resorcinol/formaldehyde molar ratio of 1:2 is preferably chosen.

The hydroxylated benzene and the aldehyde are mixed in a solvent that is readily dried in air, for example water, a cetone, an alcohol or a mixture thereof. The solvent in which the mixing takes place can be already present in the starting aldehyde or added at the time of the starting mixture.

In the case of water, the starting mixture generally has an acid pH from 2 to 4.

To increase the pH of the starting mixture, a base is used such as an alkaline or alkaline earth hydroxide that is sufficiently soluble in the solvent. Examples are NaOH, KOH, an alkaline or alkaline earth carbonate that is sufficiently soluble, such as Na₂CO₃, Li₂CO₃, K₂CO₃, tetramethyl ammonium, or any base that does not react with the monomers of the starting mixture.

An alkaline hydroxide is preferably chosen, more particularly NaOH or sodium hydroxide.

The hydroxylated benzene-aldehyde organic gel is obtained as soon as a change of viscosity is observed.

In particular, the method of preparing the organic gel of determined porous texture is characterised by adding a metal salt to the starting mixture.

The metal salt of the invention is soluble in the solution of hydroxylated benzene-aldehyde in the chosen solvent or made soluble by complexation either directly with the hydroxylated benzene or indirectly by a complexing agent. The metal salt may also be a compound comprising a metal cation such as the hexachloroplatinate cation, for example, in particular hydrogen hexachloroplatinate.

The complexing agents referred to above are, for example, an amine, a benzene ring or a heterocycle, and can more particularly be EDTA (ethylenediaminetetra-acetic acid), HEDTA (hydroxyethylethylenediaminetriacetic acid), DTPA (diethylenetriaminepentaacetic acid), or DOTA (1,4,7,10-tetraazacyclododecane-1,4,7,10-tetraacetic acid). They are added in sufficient quantity to maintain the metal complexed and dissolved in the mixture throughout the preparation of the gel.

The metal can be a metal belonging to group IVb (Ti, Zr, Hf), Vb (V, Nb, Ta), VIb (Cr, Mo, W), VIIb (Mn, Tc, Re), VIII (Fe, Co, Ni, Ru, Rh, Pd, Os, Ir, Pt), Ib (Cu, Ag, Au), or an alloy of said metals. The quantity of metal salt introduced depends on the required metal content of the material.

The aldehyde is generally added after completely dissolving the metal salt in the hydroxylated benzene to form the starting mixture. The starting pH is generally from 2 to 4 if the solvent is water.

The pH of the mixture is then raised to the required value by means of a base. If the pH of the starting solution were higher than the required pH, the pH could be adjusted using an acid such as HNO₃ or acetic acid, for example.

A second aspect of the invention consists in a method of preparing carbon material with a determined porous texture comprising drying the organic gel obtained from the starting mixture.

The solvent can be eliminated by solvent transfer, supercritical drying, or merely by evaporation of the solvent, in vacuum, in the open air, or in a flow of gas. Drying by simple evaporation is preferable. Three variables condition drying: pressure, temperature and drying environment. These depend on the required drying rate. Controlling the drying rate is important for preserving the shape of the carbon material, especially when preparing a thin plate. Drying too fast, especially at the start of the process, risks deforming the plate, especially if each face is not exposed identically to the atmosphere. To slow drying, a moist atmosphere may be used. To accelerate drying, a stream of dry gas such as nitrogen, a rare gas or dry air may be used.

Once drying has been completed, a material that is generally monolithic is obtained whose texture varies with the pH of the starting solution. The material may be very porous (specific surface area: 500 m²/g—pore volume: 1.3 cm³/g or even higher), or have a very low porosity (specific surface area: <40 m²/g—pore volume: 0.3 cm³/g or even lower).

A third aspect of the invention relates to a method of preparing carbon material of determined porous texture comprising pyrolysis of the organic gel obtained after drying.

The dried organic gel is pyrolysed in an inert atmosphere, with infiltration of oxygen prevented. In the case of materials not charged with metal, traces of oxidising gas lead to a modification of the porous texture, more specifically to an increase of the specific surface area caused by an increase in the quantity of micropores.

The maximum pyrolysis temperature may be from 500° C. to 3 000° C. It can be achieved directly by progressive heating or in successive steps at chosen staged temperatures (as a general rule, these temperatures correspond to the elimination of given compounds).

The final pyrolysis temperature may modify the final texture of the material, as described by Chuan Lin and James A. Ritter (Carbon 38 (2000), 849-861). The duration of the pyrolysis varies with the size of the materials to be pyrolysed. It is in fact sufficient to verify that the material is made up only of carbon at the end of pyrolysis.

The final carbon material obtained in this way is practically pure. Its porous texture is directly dependent on the conditions of preparation of the starting organic gel. To obtain a high specific surface area and a high pore volume, together with a wide distribution of the pore size for the resorcinol-formaldehyde mixture, the pH is preferably from 6 to 7.

All physical-mechanical properties of the material, such as the mechanical strength or hardness of the material, depend on the porous texture.

Thus for pH values from 6.0 to 6.9, a porous material is obtained that has both micropores and mesopores. If the pH is greater than or equal to 7, the material is virtually non-porous. If the pH is from 5.5 to 6.0, the material obtained becomes microporous and macroporous (with few or no mesopores) and finally loses its mechanical strength (friable material) if the pH is lower than 5.5.

In particular, the method according to the invention of preparing a carbon material is also aimed at a material charged with metal. In this case, the starting organic gel is a gel charged with metal subjected to drying by solvent elimination and preferably by evaporation of the solvent followed by pyrolysis in an inert atmosphere, in the same way as carbon materials with no metal. To modify the porous texture it is possible to add small quantities of an oxidising gas during pyrolysis.

The final pyrolysis temperature may modify the final texture of the material, as described by Chuan Lin and James A. Ritter (Carbon 38 (2000), 849-861).

The duration of the pyrolysis varies with the size of the materials to be pyrolysed. To be sure that pyrolysis has finished, it is in fact sufficient to verify that the material obtained is made up only of carbon and metal at the end of pyrolysis.

The porous texture obtained for the carbon material charged with metal is also a function of the gel preparation conditions. The range of pH variation yielding materials having in combination a high specific surface area, a large pore volume, and a high mechanical strength varies with the metal introduced and also with the complexing agent used to dissolve the metal salt.

In the case of materials charged with metal obtained from a mixture of resorcinol and formaldehyde in water, the metal is introduced into the starting solution by way of a soluble metal salt or a metal salt that has been made soluble by complexation. In this case, we have found that the beneficial pH range, i.e. that for which there is finally obtained a carbon material having a large specific surface area, a large pore volume and sufficient mechanical strength, varies with the metal introduced into the mixture, but also with the nature and the quantity of the complexing agent used.

In the case of a material charged with nickel obtained by direct complexing of nickel with resorcinol, to obtain a high specific surface area and a high pore volume as well as a wide pore size distribution for the resorcinol-formaldehyde mixture, the pH is preferably from 5.5 to 7.

For pH values from 5.5 to 6.75, a material is obtained that is both microporous and mesoporous. If the pH is higher than or equal to 7, the material is virtually non-porous. If the pH is lower than 5.5, the material obtained becomes microporous and macroporous (few or no mesopores) and loses its mechanical strength (friable material).

In the case of material charged with Pd, and with DTPA as the complexing agent, if the starting mixture has a pH from 6.25 to 7.25, a material is obtained that is both microporous and mesoporous. If the pH is greater than or equal to 7.5, the material is virtually non-porous. If the pH is lower than 6.25, the material obtained becomes microporous and macroporous (few or no mesopores) and loses its mechanical strength (friable material).

The carbon material, whether charged with metal or not, is a good electrical conductor. It is also monolithic and has a permeability to gases that varies with the porous texture of the material.

These materials are used in chemical catalysis and in electrochemistry, for example in the fabrication of fuel cell electrodes, especially electrodes for alkaline fuel cells, proton exchange membrane fuel cells and phosphoric acid fuel cells.

The invention is illustrated hereinafter by means of examples.

### EXAMPLE 1

### Preparation of Resorcinol-Formaldehyde Gels in Water

The reaction of polymerisation of the resorcinol with the formaldehyde takes place in water as solvent. The quantities of resorcinol and formaldehyde brought together are in molar proportions of 1:2 (stoichiometric proportions). The proportion of solvent can vary, but in our case was 10 ml of water per 5.27 g of resorcinol.

Reagents Used

Resorcinol: VEL—straws—pure

Formol: ALDRICH—solution of 37% by weight in water

Sodium hydroxide: VEL—pellets—98-100% pure

Deionised water

Preparation of the Gel

5.27 g of resorcinol are dissolved in 10 ml of deionised water in a sealed 100 ml flask: the mixture is agitated magnetically until the resorcinol has completely dissolved (approximately 10 minutes).

7.2 ml of formol are then added, and the mixture is homogenised by agitation.

The pH of the solution that will form the gel is then set at 6 to 7 using a basic aqueous solution (NaOH).

The solution is placed in a closed container and heated (85° C.) for three days.

The material obtained is a gel that is deep red and translucent (for the highest pH values) to light brown and opaque (for the lowest pH values).

Drying of the Gel

The gel obtained is dried simply by evaporation of the solvent. The gel is placed in an oven at 60° C. and the pressure is progressively reduced from 1.013*10⁵ Pa to 1*10³ Pa. This drying step is spread over five days. The gel is finally left in a vacuum (1*10³ Pa) at 150° C. for three days.

Once drying is completed, a monolithic material is obtained whose texture varies with the pH of the starting solution. The material can be very porous (specific surface area: 500 m²/g—pore volume: 1.3 cm³/g or even higher), or have a very low porosity (specific surface area: <40 m²/g—pore volume: 0.1 cm³/g or even lower).

Pyrolysis in an Inert Atmosphere

The dry gel is then pyrolysed at 800° C. (2 h) in an inert atmosphere (N₂). In our case, this temperature was reached via two plateaux (150° C.-15 min; 400° C.-1 h).

The material obtained in this way is made up of virtually pure carbon. Its texture also varies with the gel preparation conditions, i.e. the conditions for preparation of the starting mixture: it can be very porous (specific surface area: 600 m²/g or even higher-pore volume: 1.5 cm³/g or even higher), or have a very low porosity (specific surface area: <40 m²/g—pore volume: <0.1 cm³/g).

Consisting of carbon, the material is a good conductor of electricity. It is also monolithic and has a permeability to gases that varies greatly with the texture of the material, from virtually non-permeable to more than 2 ml/(min.cm²), for a permeability test conducted with nitrogen, and a pressure difference of 5.33*10⁴ Pa between the two sides of a 1 mm thick plate.

After drying and pyrolysis, gel volume was compacted by an amount varying from 60 to 80% according to the envisaged pH, but retained its original shape (carbon material geometrically similar to the starting gel). This compaction can be provided at the start and the size of the moulded part adapted accordingly.

Preparation Conditions

The preparation conditions of the gel, and to be more precise the pH of the starting resorcinol-formaldehyde solution, determined the texture of the final carbon material, i.e. the specific surface area, the pore volume and the pore size distribution. Table 1 sets out the textural properties of the carbon materials obtained for pH values varying from 7.05 to 6.03.

TABLE 1
specific surface area and pore volume of carbon
materials (after pyrolysis) for which the pH of the
starting mixture varies from 7.05 to 6.03.

| | Specific | Micropore | Total pore |
|---|---|---|---|
| | surface area | volume | volume |
| Starting pH | (m²/g) | (cm³/g) | (cm³/g) |
| 6.03 | 450 | 0.18 | 0.2* |
| 6.47 | 560 | 0.24 | 1.1* |
| 6.59 | 560 | 0.24 | 0.9* |
| 6.78 | 526 | 0.23 | 1.1 |
| 6.89 | 526 | 0.23 | 0.5 |
| 7.05 | <40 | <0.1 | <0.1 |

Specific surface area: determined by the BET method.
Micropore volume: pore volume determined by the Dubinin method representing pores smaller than 2 nm.
Total pore volume: pore volume determined from the volume adsorbed at saturation, representing pores smaller than 50 nm.
*material having pores larger than 50 nm and whose contribution to the pore volume was not taken into account.

For pH values from 6.0 to 6.9, a material is obtained that is both microporous and mesoporous. If the pH is greater than or equal to 7, the material is virtually non-porous. If the pH is from 5.5 to 6.0, the material obtained becomes microporous and macroporous (few or no mesopores) and finally loses its mechanical strength (friable material) if the pH is lower than 5.5.

### EXAMPLE 2

### Preparation of Resorcinol-Formaldehyde Gels Charged with Nickel

In exactly the same way as for example 1, the reaction of polymerisation of the resorcinol with the formaldehyde takes place in water with additionally tetrahydrated nickel acetate: ALDRICH—99.998%.

The quantities of resorcinol and formaldehyde brought together are in molar proportions of 1:2 (stoichiometric proportions). The proportion of solvent can vary, but in our case was 10 ml of water per 5.27 g of resorcinol.

The metal (Ni) is introduced in the form of a salt (nickel acetate). The quantity of salt varies as a function of the required metal content of the final carbon material. Here, the quantity of nickel acetate tetrahydrate introduced was 0.191 g per 10 ml of water (and 5.27 g of resorcinol).

The solution of resorcinol in water is formed first, and the metal salt added to it. The resorcinol complexes the nickel (yielding a dark yellow solution). The mixture is agitated mechanically for several hours. The formol is added after complete dissolution of the salt and complexing of the metal.

The pH of the solution that will form the gel is then set at 5.5 to 7 using a basic aqueous solution (NaOH).

The solution is placed in a closed container and heated (85° C.) for three days.

The material obtained is a gel that is deep red and translucent (for the highest pH values) to light brown (for the lowest pH values).

Drying of the Gel

The gel obtained is dried simply by evaporation of the solvent. The gel is placed in an oven at 60° C. and the pressure is progressively reduced from 1.032*10⁵ Pa to 1*10³ Pa. This drying step is spread over five days. The gel is finally left in a vacuum (1*10³ Pa) at 150° C. for three days.

Once the drying is completed, there is obtained a monolithic material whose texture varies with the preparation conditions. The material can be very porous (specific surface area: 500 m²/g—pore volume: 1.3 cm³/g or even higher), or have a very low porosity (specific surface area: <40 m²/g—pore volume: <0.3 cm³/g).

Pyrolysis in an Inert Atmosphere

The dry gel is then pyrolysed at 800° C. (2 h) in an inert atmosphere (N₂). In our case, this temperature was reached via two plateaux (150° C.-15 min; 400° C.-1 h).

The material obtained in this way is made up of virtually pure carbon within which is dispersed the metal in the reduced state (metallic Ni). Its texture also varies with the gel synthesis conditions: it can be very porous (specific surface area: 700 m²/g or even higher—pore volume: 1.5 cm³/g or even higher), or have a very low porosity (specific surface area: <40 m²/g—pore volume: <0.1 cm³/g).

Consisting of carbon, the material is a good conductor of electricity. It is also monolithic and has a permeability to gases that again varies greatly with the texture of the material (variation of the same order of magnitude as the material not charged with metal).

After drying and pyrolysis, the gel was compacted by 60 to 80% (variable according to the starting pH), but retained its original shape (carbon material geometrically similar to the starting gel). This compaction can be provided at the start and the size of the moulded part adapted accordingly.

Gel Preparation Conditions

The gel preparation conditions, to be more precise the pH of the starting resorcinol-formaldehyde solution, determined the porous texture of the final material, i.e. the specific surface area, the pore volume and the pore size distribution.

Table 2 gives the specific surface area and pore volume of the carbon materials charged with nickel (approximately 1.5% by weight) obtained for pH values varying from 6 to 7.

TABLE 2
specific surface area and pore volume of carbon
materials charged with Ni for which the gel preparation
pH varies from 6 to 7.

| | Specific | Micropore | Total pore |
|---|---|---|---|
| | surface area | volume | volume |
| Starting pH | (m²/g) | (cm³/g) | (cm³/g) |
| 6.00 | 682 | 0.29 | 1.5 |
| 6.23 | 705 | 0.30 | 1.2 |
| 6.57 | 606 | 0.27 | 1.0 |
| 6.75 | 567 | 0.25 | 0.7 |
| 7.00 | 145 | 0.07 | 0.15 |

Specific surface area: determined by the BET method.
Micropore volume: pore volume determined by the Dubinin method representing pores smaller than 2 nm.
Total pore volume: pore volume determined from the volume adsorbed at saturation, representing pores smaller than 50 nm.

For pH values from 6 to 6.75, a material is obtained that is both microporous and mesoporous. If the pH is greater than or equal to 7, the material is virtually non-porous. If the pH is less than 6, the material obtained becomes microporous and macroporous (few or no mesopores) and finally loses its mechanical strength (friable material).

### EXAMPLE 3

### Preparation of Resorcinol-Formaldehyde Gels Charged with Palladium

In exactly the same way as for example 1, the reaction of polymerisation of the resorcinol with the formaldehyde takes place in water and additionally with palladium acetate (ALDRICH—98%) and a complexing agent, diethylenetriaminepentaacetic acid (ALDRICH—98%).

The quantities of resorcinol and formaldehyde brought into contact are in molar proportions of 1:2 (stoichiometric proportions). The proportion of solvent can vary, but in our case was 10 ml of water per 5.27 g of resorcinol.

The metal (Pd) is introduced in the form of a salt (palladium acetate). The quantity of the salt varies as a function of the required metal content of the final carbon material. Here, the quantity of palladium acetate introduced was 0.0970 g per 10 ml of water (and 5.27 g of resorcinol). The palladium is complexed by DTPA (diethylenetriaminepentaacetic acid) and the complexing agent/metal molar ratio is 1, which means that 0.1700 g of DTPA are introduced into the solution.

The solution of complexing agent and metal salt in water is formed first and the resorcinol added. The mixture is agitated mechanically for several hours. A clear orange solution is obtained. The formol is added after complete dissolution of the salt and complexing of the metal.

The pH of the solution that will form the gel is then set to 5 to 8 using a basic aqueous solution (NaOH).

The solution is placed in a closed container and heated (85° C.) for three days.

The material obtained is a gel that is deep red and translucent (for the highest pH values) to light brown and opaque (for the lowest pH values).

Drying of the Gel

The gel obtained is dried simply by evaporation of the solvent. The gel is placed in an oven at 60° C. and the pressure is progressively reduced from 1.013*10⁵ Pa to 1*10³ Pa. This drying step is spread over five days. The gel is finally left in a vacuum (1*10³ Pa) at 150° C. for three days.

Once drying is completed, a monolithic material is obtained whose texture varies with the synthesis conditions. The material can be very porous (specific surface area: 500 m²/g—pore volume: 1.3 cm³/g or even higher), or have a very low porosity (specific surface area: <100 m²/g—pore volume: <0.3 cm³/g).

Pyrolysis in an Inert Atmosphere

The dry gel is then pyrolysed at 800° C. (2 h) in an inert atmosphere (N₂). In our case, this temperature was reached via two plateaux (150° C.-15 min; 400° C.-1 h).

The material obtained in this way is made up of virtually pure carbon within which is dispersed the metal in the reduced state (metallic Pd). Its texture also varies with the gel synthesis conditions: it can be very porous (specific surface area: 600 m²/g or even higher-pore volume: 1.4 cm³/g or even higher), or have a very low porosity (specific surface area: <40 m²/g—pore volume: <0.3 cm³/g).

Consisting of carbon, the material is a good conductor of electricity. It is also monolithic and has a permeability to gases that again varies greatly with the texture of the material (variation of the same order of magnitude as the material not charged with metal).

After drying and pyrolysis, the gel was compacted by 60 to 80% (variable according to the starting pH), but retained its original shape (carbon material geometrically similar to the starting gel). This compaction can be provided at the start and the size of the moulded part adapted accordingly.

Gel Preparation Conditions

The preparation conditions of the gel, to be more precise the pH of the starting resorcinol-formaldehyde solution (containing the complexed metal), determined the porous texture of the final material, i.e. the specific surface area, the pore volume and the pore size distribution.

Table 3 gives the specific surface area and pore volume of the carbon materials charged with palladium (approximately 1.5% by weight) obtained for pH values varying from 5.53 to 7.49.

TABLE 3
specific surface area and pore volume of carbon
materials for which the gel preparation pH varies from
5.53 to 7.49.

| | Specific | Micropore | Total pore |
|---|---|---|---|
| | surface area | volume | volume |
| Starting pH | (m²/g) | (cm³/g) | (cm³/g) |
| 5.53 | 607 | 0.26 | 1.43* |
| 6.03 | 567 | 0.25 | 1.16* |
| 6.64 | 546 | 0.24 | 1.12 |
| 7.01 | 508 | 0.22 | 0.656 |
| 7.49 | <40 | <0.1 | <0.3 |

Specific surface area: determined by the BET method.
Micropore volume: pore volume determined by the Dubinin method representing pores smaller than 2 nm.
Total pore volume: pore volume determined from the volume adsorbed at saturation, representing pores smaller than 50 nm.
*material having pores larger than 50 nm and whose contribution to the pore volume was not taken into account.

For pH values from 6.25 to 7.25, a material that is both microporous and mesoporous is obtained. If the pH is greater than or equal to 7.5, the material is virtually non-porous. If the pH is less than 6.25, the material obtained becomes microporous and macroporous (few or no mesopores) and finally loses its mechanical strength (friable material).

### EXAMPLE 4

### Preparation of Resorcinol-Formaldehyde Gels Charged with Platinum

In exactly the same way as for example 1, the reaction of polymerisation of the resorcinol with the formaldehyde takes place in water and additionally with hydrogen hexachloroplatinate (hydrated—ALDRICH—99.9%) and a complexing agent, diethylenetriaminepentaacetic acid (ALDRICH—98%).

The quantities of resorcinol and formaldehyde brought into contact are in molar proportions of 1:2 (stoichiometric proportions). The proportion of solvent can vary, but in our case was 10 ml of water per 5.27 g of resorcinol.

The metal (Pt) is introduced by adding hydrogen hexachloroplatinate, the quantity of which varies as a function of the required metal content of the final carbon material. Here, the quantity of hydrogen hexachloroplatinate introduced was 0.0966 g per 10 ml of water (and 5.27 g of resorcinol). The platinum is complexed by DTPA (diethylenetriaminepenta acetic acid) and the complexing agent/metal molar ratio is fixed at 2, which means that 0.1753 g of DTPA are introduced into the solution.

The solution of complexing agent and hydrogen hexachloroplatinate in water is formed first, to which resorcinol is then added. Hydrogen hexachloroplatinate is very soluble in water, but the complexing agent is nevertheless added to prevent premature reduction of the platinum by the formaldehyde and thus precipitation of the metal before the gel sets. The mixture is agitated mechanically for several hours. A clear orange solution is obtained. The formol is then added.

The pH of the solution is then set at 5 to 8 using a base (NaOH).

The solution is placed in a closed container and heated (85° C.) for three days.

The material obtained is a gel that is deep red and translucent (for the highest pH values) to light brown and opaque (for the lowest pH values).

Drying of the Gel

The gel obtained is dried simply by evaporation of the solvent. The gel is placed in an oven at 60° C. and the pressure is progressively reduced from 1.013*10⁵ Pa to 1*10³ Pa. This drying step is spread over five days. The gel is finally left in a vacuum (1*10³ Pa) at 150° C. for three days.

Once the drying is completed, a monolithic material is obtained whose texture varies with the synthesis conditions. The material can be very porous (specific surface area: 400 m²/g—pore volume: 1.5 cm³/g or even higher), or have a very low porosity (specific surface area: <100 m²/g—pore volume: 0.3 cm³/g or even lower).

Pyrolysis in an Inert Atmosphere

The dry gel is then pyrolysed at 800° C. (2 h) in an inert atmosphere (N₂). In our case, this temperature was reached via two plateaux (150° C.-15 min; 400° C.-1 h).

The material obtained in this way is made up of virtually pure carbon within which is dispersed the metal in the reduced state (metallic Pt). Its texture also varies with the gel synthesis conditions: it can be very porous (specific surface area: 500 m²/g—pore volume: 1.5 cm³/g or even higher), or have a very low porosity (specific surface area: <100 m²/g—pore volume: <0.3 cm³/g or even lower).

Consisting of carbon, the material is a good conductor of electricity. It is also monolithic and has a permeability to gases that again varies greatly with the texture of the material (variation of the same order of magnitude as the material not charged with metal).

After drying and pyrolysis, the gel was compacted by from 60 to 80% (variable according to the starting pH), but retained its original shape (carbon material geometrically similar to the starting gel). This compaction can be provided at the start and the size of the part adapted accordingly.

Gel Preparation Conditions

The preparation conditions of the gel, to be more precise the pH of the starting resorcinol-formaldehyde solution (containing the complexed metal), determined the porous texture of the final material, i.e. the specific surface area, the pore volume and the pore size distribution.

Table 4 gives the specific surface area and pore volume of the carbon materials charged with platinium (approximately 1.5% by weight) obtained for pH values varying from 7.00 to 7.80.

TABLE 4
specific surface area and pore volume of carbon
materials (after pyrolysis) for which the gel preparation
pH varies from 7.00 to 7.80.

| | Specific | Micropore | Total pore |
|---|---|---|---|
| | surface area | volume | volume |
| Starting pH | (m²/g) | (cm³/g) | (cm³/g) |
| 7.00 | 495 | 0.20 | 0.25* |
| 7.25 | 550 | 0.24 | 1.13* |
| 7.50 | 350 | 0.15 | 0.41 |
| 7.80 | <40 | <0.1 | <0.1 |

Specific surface area: determined by the BET method.
Micropore volume: pore volume determined by the Dubinin method representing pores smaller than 2 nm.
Total pore volume: pore volume determined from the volume adsorbed at saturation, representing pores smaller than 50 nm.
*material having pores larger than 50 nm and whose contribution to the pore volume was not taken into account.

For pH values from 7 to 7.5, a material is obtained that is both microporous and mesoporous. If the pH is greater than or equal to 7.8, the material is virtually non-porous. If the pH is from 6.5 to 7, the material obtained becomes microporous and macroporous (few or no mesopores) and finally loses its mechanical strength (friable material) if the pH is less than 6.5.

### EXAMPLE 5

### Application to Catalysis

Catalysts containing nickel, palladium or platinum synthesised by the method explained in examples 2, 3 and 4 have been tested for the reaction of hydrogenation of ethylene in a test installation. The catalysts tested were synthesised either by direct introduction of the metal (nickel, palladium and platinum) into the gel during synthesis or by impregnation of porous carbon (platinum only). The test installation comprises principally three gas lines for the ethylene, argon and hydrogen, whose flowrates are adjusted with the aid of mass flowmeters, a tubular reactor (diameter=8 mm; length=20 cm) placed in a pulsed air oven, and a chromatograph to analyse the gaseous effluent. The temperature of the oven is regulated and is measured with the aid of a thermocouple slid into a stainless steel sheath dipping into a catalytic bed. Because of pressure drop, the total pressure in the reactor is slightly higher than atmospheric pressure.

If the conditions are such that the reactor can be regarded as a differential reactor (homogeneous reagent concentrations over the entire length of the reactor), i.e. if the rate of conversion of the reagents into products is low (of the order of 10% maximum), it is shown that there exists a relationship between the conversion rate (f) and the apparent activation energy (Eₐ) of the following type:

$$ln f = ln C - \frac{{E}_{a}}{R T}$$

where C is a constant, R is the perfect gas constant and T is the temperature.

Analysing the gaseous effluent and thereby determining the conversion rate therefore measures the apparent activation energy of the metal catalyst.

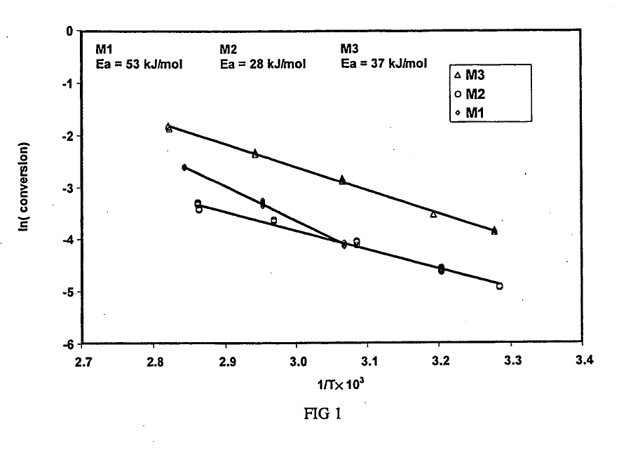

FIG. 1 shows catalytic test results obtained for hydrogenation of the ethylene and the measured apparent activation energy in the case of nickel (M1), palladium (M2) and platinum (M3), all three dispersed on carbon by the method of the invention.

The catalytic tests, carried out at temperatures from 30° C. to 80° C. after complementary processing of reduction of the metal with hydrogen (320° C.-3 hours), show that the nickel, the palladium and the platinum dispersed on carbon supports by the method of the invention are active for hydrogenation of ethylene. The apparent activation energy (Eₐ) measured for nickel, palladium and platinum corresponds to the standard values found in the literature. Eₐ=53 kJ/mol, Eₐ=28 kJ/mol and Eₐ=44 kJ/mol were respectively measured for nickel, palladium and platinum. These results prove that the metal is accessible to the reagents and is catalytically active.

### EXAMPLE 6

### Application to Electrochemistry

A few preliminary tests have been carried out with the aim of showing that the materials obtained have an electrocatalytic activity. Two samples of porous carbon synthesised by the sol-gel process using the method explained for example 1 were produced in the form of monolithic discs 1 mm thick and with a surface area of 5 cm². The principal characteristics of the porous texture of these samples are set out in Table 5.

TABLE 5
textural characteristics of the electrodes
tested.

| | | | | | Content by |
|---|---|---|---|---|---|
| | | S_{BET} | V_{DUB} | V_{P} | weight of Pt × |
| Sample | | m²/g | cm³/g | cm³/g | 10⁻⁴ |
| | 1 | 302 | 0.14 | 1.33 | 1 |
| | 2 | 442 | 0.2 | 0.9 | 1.5 |

The samples were covered with a 15 nm thickness of platinum on one face by plasma metallization in a vacuum. The average platinum content obtained is given in Table 5. The samples were fitted as electrodes in a potentiometer circuit with a basic electrolyte (1M NaOH), a graphite counter-electrode and an Ag/AgCl reference electrode. The working electrode was mounted so that it was in contact with the electrolyte on one face and fed with gaseous hydrogen at a pressure of 1.1 atm on the other face.

The current density of the anodically polarized working electrode under steady state conditions was measured as a function of the overvoltage. The electrode 1 whose platinised face was on the hydrogen supply side gave a current density of 20 mA/cm² for an overvoltage of 0.5 V. The electrode 2 whose platinised face was on the electrolyte side gave a current density of 18 mA/cm² for an overvoltage of 0.7 V. For both electrodes, the overvoltage evolves practically in proportion to the current density up to the values mentioned. Stable conditions can no longer be maintained for higher current densities.

1. A method of preparing an organic gel with a determined porous texture comprising mixing hydroxylated benzene with an aldehyde in a solvent to form a gel, which method is characterised in that the texture of the gel is regulated by adjusting the pH of the starting mixture.

2. A preparation method according to claim 1, characterised in that the pH is adjusted by means of a base.

3. A preparation method according to the preceding claim, characterised in that the base is NaOH.

4. A preparation method according to claim 1, characterised in that the hydroxylated benzene is resorcinol and the aldehyde is formaldehyde.

5. A preparation method according to the preceding claim, characterised in that the resorcinol and the formaldehyde are mixed in molar proportions of 1:2.

6. A preparation method according to claim 1, characterised in that a metal salt is added to the starting mixture.

7. A preparation method according to the preceding claim, characterised in that a complexing agent is also added to the starting mixture.

8. A preparation method according to claim 6, characterised in that the metal is nickel.

9. A preparation method according to claim 1 of preparing an organic gel, further comprising a step of drying the gel obtained.

10. A preparation method according to the preceding claim of preparing an organic gel, characterised in that the drying is effected simply by evaporation of the solvent.

11. A preparation method according to claim 9 of preparing an organic gel, characterised in that the organic gel is charged with metal.

12. A method of preparing carbon material with a determined porous texture, comprising pyrolysis of a dry organic gel obtained by a preparation method according to either claim 9.

13. A method according to claim 12 of preparing a carbon material, characterised in that the carbon material is charged with metal.

14. An organic gel charged with metal and with a determined porous texture, obtained by the preparation method of claim 6.

15. An organic gel charged with metal, obtained by the preparation method according to claim 11.

16. A carbon material charged with metal and with a determined porous texture, obtained by the preparation method of claim 13.

17. Use of the carbon material according to claim 16 in chemical catalysis.

18. Use of the carbon material according to claim for the fabrication of fuel cell electrodes.
